(19) United States
(12) Patent Application Publication
(10) Pub. No.: US 2004/0185121 A1
(43) Pub. Date: Sep. 23, 2004

(54) Chinese herbal medicine composite system for use in treatment of paralysis

(76) Inventor: Li Cheng Yu, Taipei (TW)

Correspondence Address: LI Cheng Yu, PO Box 82-144, Taipei, TW

(21) Appl. No.: 10/393,468

(22) Filed: Mar. 21, 2003

(51) Int. Cl. A61K 35/78

(52) U.S. Cl. 424/725; 424/756

(57) ABSTRACT

This innovation is a Chinese herbal medicine composite system for use in treatment of paralysis. Within the system different formulas are prescribed according to the body condition of the patient; the system comprising three separate Chinese herbal medical formulas; the first formula to be used in treatment of patients with paralysis caused by viral infection, trauma, abnormal cellular differentiation, secondary cause induced paralysis of Exterior Yang Deficiency with Cold Stagnation type; the second prescription to be used in treatment of patients suffering paralysis of Kidney Yang Deficiency with Cold Stagnation type; and the third prescription to be used to treat patients with paralysis of Blood and Qi Deficiency combined with Yang Deficiency type. Therefore, when only one particularly type (of syndrome) is manifest throughout the course of the treatment, one single prescription can be utilized for the duration of treatment. If during the course of the treatment the patient's condition should change from one type to another, the prescription should be changed correspondingly, in order to suitably treat the paralytic patient.

### 1. FIELD OF THE INVENTION

This innovation falls under the jurisdiction of the domain of techniques and technology suitable for the therapeutic treatment of paralysis, and, through the use of three separate and unique Chinese medicinal herbal formulas can be used to control paralysis throughout the various stages of the disease, with the aim of curing the paralysis.

### 2. DESCRIPTION OF THE PRIOR ART

It should be noted that traditional Chinese medicine is an area of extremely deep and refined scholarly research, with it the roots of its theory dating back to the classic, The Book of Changes. The Book of Changes expounds on the principals of the celestial bodies and all forms of life, and can therefore be aptly referred to as the Classic of the classics.

Owing to this, the foundations and history of traditional Chinese medicine are as old as the Book of Changes, and takes the observation of the changes in the natural world as a basis for understanding the principles of disease—the body is considered to be a miniature world, a miniature universe. The Book of Changes emphasizes the importance of atmospheric and climatic changes, which in turn influence the health of the human body.

Western medicine is founded on the basis of the theory and research of western science, that is, mathematics and the natural sciences of physics, chemistry, and biology. Western medicine regards the individual as an object, and has a very short history of just over 200 years, which when compared with the thousands of years of history that Chinese medicine can claim, really does seem extremely short.

The phenomena of nature, including the orbiting of the stars, all have in common a predisposition for a state of equilibrium, which if lost, becomes chaos. This state may be referred to as a state of equilibrium in motion. Western medicine uses medicine to kill bacteria. This is very different from the methods of Chinese medicine, which is to correct states of disequilibrium

The human body is governed by the interaction if the Five Phases. The heart relates to fire, the lungs to metal, the kidneys to water, and the liver and stomach to wood and earth respectively. A healthy equilibrium is maintained in the body through the regulation of the cycles of production and restraint. Through the cycle of production the body is kept strong and healthy, as wood engenders fire, fire engenders earth, earth engenders metal, metal engenders water and water engenders wood. In such a way, the state of healthy equilibrium is maintained in the body.

If the action of restraint is over-expressed, the body is apt to suffer and become weak, as metal overacts on wood, causing damage related to both wood and metal, wood overacts on earth, earth overacts on water, water overacts on fire, and fire overacts on wood etc., all having detrimental effects on health.

At present, western medicine neurologists take the viewpoint that once the brain, spinal cord, or nerve cells have suffered damage, hemorrhage, embolism, or any other cause leading to ischemia, atrophy or necrosis, there is no way to instigate re-growth, nor any medicine which can sustain or return function.

The discoverer of this innovation is in no way in agreement with the western medical view cited above, and as a result of extensive clinical experimentation has discovered the fact that cerebral and spinal nerve cells, having suffered damage and necrosis, can re-generate and self-repair, enabling return to normal conduction, command, control and behavior.

The discoverer of this innovation attests to the innovation's concrete theory and efficacy. It should also be noted that during the course of the therapy, from having absolutely no feeling in the limbs and body right through to having completely normal function of movement, the patient will experience various changes in sensation. Such changes in perceived sensation should be regarded as optimistic signposts on the road to recovery. They progress like the waves of an ever advancing tide, as the self-healing progresses and function and ability return, fulfilling the role of testing the extent of the process and standing as proof that the nerves are still alive.

It is lamentable that there is presently no western medicine that can aid the repair of damaged nerves or instigate their complete regeneration thereby improving function. At the same time, western medicine also mistakenly identifies the aforementioned sensations experienced by the patient as abnormal phenomena, and attempts to control them through the use of strong anti-inflammatory drugs such as corticosteroids, which only hasten the atrophy of the spinal nerve cells, or blockers, which impede the brain's recognition of the spinal nerve injury. All that is left to do is to wait in vain for aid, allowing those cells still with hope of recovery to lose their window of opportunity and steadily atrophy towards a state of complete necrosis. On the other hand another common approach of western medicine, and equally mistaken, is to operate on the patient, inserting metal rods for stabilization, leading to:

1. Interruption of nerve transmission caused by anesthetic.

2. Operative hemorrhage causing impaired local blood supply, can allow the spinal nerve cells already in a state of acute hypoxia to further suffer ischemia and hypoxia, leading to irreversible and permanent damage.

3. Direct damage to spinal nerve cells and fibers suffered during the operation.

Still, the fruit of this innovator's many years research in Chinese medicine, have shed light on the symptom patterns and effective formulas for each type of spinal nerve injury-induced paralysis. It has been discovered that their body type condition in the main fall under the Chinese medical category of Kidney Yang deficiency, particularly in the post-operative and post-acute stages—which fall under the combined pattern of Kidney Yang deficiency coupled with Cold Stagnation. All cases of paralysis may be classified into the five types below:

1. Heat Stagnation type: only in the acute stage following bone fracture, edema stage. In this phase, it is suitable to use Zhi Bo Di HuangDecoction, Fu Yuan Huo Xue decoction, Dilong Powder, Jia-wei Er-Miao Powder, or other such formulas, adding individual herbs such as ruxiang, moyao, chuanqi, niuxi, sheng-duzhong, xuduan, suibu, wujia-pi, and dibie.

2. Cold stagnation type: use formula as above, or Tao Hong Si-wu Decoction or Guizi Fuling Pill, in either case further adding Si-Wu Decoction, and ruxiang, moyao, cinnamon bark mahuang, dilong and lo-rong, or other such suitable herbs.

3. Qi and Blood deficiency in combination with Yang Qi Deficiency: Use either Shi Quan Da Bu Pill or Gui Qi Jian Zhong Decoction in combination with Si Wu Decoction, further adding quanqi, cinnamon bark, mahuang, duzhong, or other such suitable herbs.

4. Qi Deficiency, Cold Stagnation with Yang Deficiency: Use either Bu Yang Huan Wu Decoction, Hui Yang Sheng Wian Decoction, Yi Qi Huo Xue Xiao Fu Decoction, Coronary Heart Formula 1 or Coronary Heart Formula 2, together with Si Wu Decoction, cinnamon bark mahuang, quanqi, lu-rong, dilong or other such suitable herbs.

5. Kidney Yang Deficiency with Cold Stagnation: It is suitable to use Shen Qi Pill, You Gui Yin (Brew), adding dried ginger, quanqi, ginseng, mahuang, or other such suitable herbs.

The process of assessing patients' conditions and allocating them according to the above stated five categories, and composing the most suitable formula for the patient is an extremely complex task.

In view of the above stated, the errors of western medical therapy in the treatment of paralysis patients may lead to irreparable injury, leaving no hope of recovery. Traditional Chinese medical therapy must depend on the allocation of the paralysed patient's condition into one of five categories, whereupon a therapeutic formula can be prescribed accordingly. The task of understanding the therapeutic process and the patient is at once challenging and perplexing.

In view of this, the innovator, relying on many years experience and research in Chinese herbal therapy, Chinese medical theory and practice in the therapy of paralysis, and after many years clinical experimentation, has succeeded in developing a Chinese herbal medicine composite system for use in the treatment of paralysis, which is reliable in the treatment of paralysed patients.

### SUMMARY OF THE INVENITON

The innovator utilized the precepts of traditional Chinese medical diagnosis to develop a system wherein patients presenting with paralysis could be divided into three separate groups, namely: Exterior Yang Deficiency with Cold Stagnation, Kidney Yang Deficiency with Cold Stagnation, Qi and Blood Deficiency with Yang Deficiency, and treated with formulas corresponding to the presenting type.

The innovator's main aim is to provide a therapy of wide ranging utility for patients suffering paralysis, and to this end presents formula 1, formula 2 and formula 3. Formula 1 should be used for treatment of paralysis caused by viral infection, external injury, or abnormal cellular differentiation or secondary induced paralysis in patients presenting with symptom patterns of Exterior Yang Deficiency with Cold Stagnation. Formula 2 should be used for treatment of patients presenting with symptom patterns of Kidney Yang Deficiency with Cold Stagnation. Formula 3 should be used for treatment of patients presenting with symptom patterns of Qi and Blood Deficiency with Yang Deficiency.

Formula 1: formula 1 comprises: mahuang 7.5˜15 g, cinnamon bark 11.25˜18.75 g, huangqin 11.25 g, honghua 11.25 g, dilong 11.25˜18.75 g, danggui tails 11.25 g, taoren paste 11.25 g, gancao 11.25 g, chuanxiong 11.25 g, chishao 11.25 g, bei huangqi 37.5 g, ruxiang 11.25 g, moyao 11.25 g, dried ginger 11.25˜18.75 g, tianxiong 11.25˜18.75 g, and chuanqi powder 11.25 g. This formula is for use in treatment of paralysis caused by viral infection, external injury, or abnormal cellular differentiation or of secondary induced paralysis in patients presenting with symptom patterns of Exterior Yang Deficiency with Cold Stagnation.

Formula 2: formula 2 comprises dry dihuang 15 g, shan-zhuyu, 15 g, huai-shanyao 15 g, bai-fuling 15 g, tusi-zi 15 g, huai-niuxi 15 g, danggui 15 g, lurong 15 g, gouji-zi 11.25 g, fried duzhong 11.25 g, mahuang 11.25 g, huangbo 11.25 g, dried ginger 18.75 g˜26.25 g, tianxiong 18.75˜26.25 g, and cinnamon bark 18.25 g. This formula is suitable for use in the treatment of paralysis where symptoms of low blood pressure, osteoporosis, atrophy of the liver, atrophy of the kidney, coldness in the limbs, low body temperature, and or constipation form a pattern of Kidney Yang Deficiency with Cold Stagnation.

Formula 3: formula 3 comprises ginseng 11.25 g, fried baizhu 11.25 g, bai-fuling 11.25 g, gancao 11.25 g, danggui 11.25 g, chuanxiong 11.25 g, chi-shaoyao 11.25 g, gan-dihuang 11.25 g, bei-huangqi 37.5˜75 g, cinnamon bark 11.25˜18.75 g, dried ginger 11.25˜30 g, tianxiong 11.25˜30 g, mahuang 7.5˜15 g, dilong 11.25˜18.75 g, huangbo 11.25 g, lurong 11.25˜18.75 g and powdered chuanqi 11.25 g. This formula is suitable for use in the treatment of paralysis in patients where symptoms of poor appetite, hemiparalysis and impaired hematopoiesis form a pattern of Qi and Blood Deficiency with Yang Deficiency.

If throughout the course of the disease (from onset to cure), the patient presents with just one symptom-pattern, then accordingly, only one formula need be administered to effect a complete cure. If however the patient's symptoms change during the course of the illness from one pattern to another, then the formula prescribed should be changed correspondingly, in order to effect a safisfactory cure.

In order to allow the members of the committee to better understand the method of preparation as well as the distinctive features of the innovation, below is cited a more practical and precise explanation of the innovation, giving concrete and practical information on the technical details of the aforementioned:

### DETAILED DESCRIPTION OF THE PREFERRED EMBODIMENT

The innovation is a Chinese herbal medicine composite system for use in treatment of paralysis, comprising herbal formulas formula 1, formula 2 and formula 3, formula 1 being suitable for treatment of paralysis caused by viral infection, external injury, or abnormal cellular differentiation or secondary induced paralysis in patients presenting with symptom patterns of Exterior Yang Deficiency with Cold Stagnation, formula 2 being suitable for treatment of paralysis in patients presenting with symptom patterns of Kidney Yang Deficiency with Cold Stagnation, and Formula 3 being suitable for treatment of paralysis in patients presenting with symptom patterns of Qi and Blood Deficiency with Yang Deficiency.

The preparation of the Chinese herbal formulas constituting this innovation is detailed as follows:

Formula 1: formula 1 comprises: mahuang 7.5˜15 g, cinnamon bark 11.25˜18.75 g, huangqin 11.25 g, honghua 11.25 g, dilong 11.25˜18.75 g, danggui tails 11.25 g, taoren paste 11.25 g, gancao 11.25 g, chuanxiong 11.25 g, chishao 11.25 g, bei huangqi 37.5 g, ruxiang 11.25 g, moyao 11.25 g, dried ginger 11.25˜18.75 g, tianxiong 11.25˜18.75 g, and chuanqi powder 11.25 g. This formula is for use in treatment of paralysis caused by viral infection, external injury, or abnormal cellular differentiation or of secondary induced paralysis in patients presenting with symptom patterns of Exterior Yang Deficiency with Cold Stagnation.

The method of preparation for above mentioned formula is as follows: The mahuang, cinnamon bark, huangqin, danggui tails, chishao and tianxiong should all be cut into small pieces or grains 0.1˜0.3 cm in diameter using a suitable herb cutting machine. The dilong should be cut into lengths of 1.5 cm. The huangqi and dried ginger should be sliced into pieces 0.15 thick. The chuanqi should be machine-ground to a fine powder. The gancao should be sliced into pieces 0.15 in thickness. The ruxiang and moyao should be crystal-cut into 1 cm³ cubes. The above ingredients, excluding the powdered chuanqi, along with an amount of water equivalent to 8 rice-bowlsful, should be entered into a pot and brought to the boil, and subsequently simmered for 1˜1.5 hours. When the amount of water has reduced to the equivalent of 3 bowlsful, the powdered chuanqi should be added. The resulting mixture should then be divided into three equal portions, each constituting one dose of the medicine—to be administered cold, thrice daily—one dose to be administered at 8 am, before food, another at 3 pm and the third in the evening before bed. If required, ginseng may be added to the formula, at a dosage of 11.25 g per day, ideally taken by masticating for some time before swallowing, otherwise being ground to a fine powder and being added to the prepared decoction along with the chuanqi in the above stated manner. Extra cinnamon bark may also be ground to a fine powder and mixed into the already reduced decoction, without being cooked.

Formula 2: formula 2 comprises dry dihuang 15 g, shan-zhuyu, 15 g, huai-shanyao 15 g, bai-fuling 15 g, tusi-zi 15 g, huai-niuxi 15 g, danggui 15 g, lurong 15 g, gouji-zi 11.25 g, fried duzhong 11.25 g, mahuang 11.25 g, huangbo 11.25 g, dried ginger 18.75 g˜26.25 g, tianxiong 18.75˜26.25 g, and cinnamon bark 18.25 g. This formula is suitable for use in the treatment of paralysis where symptoms of low blood pressure, osteoporosis, atrophy of the liver, atrophy of the kidney, coldness in the limbs, low body temperature, and or constipation form a pattern of Kidney Yang Deficiency with Cold Stagnation.

The method of preparation for formula 2 is as follows: the dihuang, huai-shanyao, bai-fuling, danggui, crude lurong and dried ginger should all be machine-cut to a thickness of 0.15 cm. The huai-niuxi should be cut into lengths of 1 cm. The duzhong should first be fried lightly; frying only until it starts to give off a pungent aroma and white smoke. The duzhong should then be immersed in salt water, and left until the water has soaked right into the material, thereupon being retrieved from the water and fried on a low heat until dry to the touch. Consequently the duzhong should be placed in an oven at 50° C., until completely dry. The danggui should be sprayed or dipped in rice wine, which should be allowed to soak right through the tissue of the material. The danggui should then be sliced into strips which are then to be placed in a low temperature oven (40° C.), until the moisture has evaporated. A pestle and mortar should be used to smash the outer skin of the gouji-zi. The mahuang, tianxiong, cinnamon bark and huangbo should all be machine-ground into grains of 0.1˜1.5 cm in diameter. The tusi-zi should be stir-fried until the outer casing bursts open. The shan-zhuyu should first be washed and dried, whereupon the flowers, seeds and stems should be removed. Finally all the ingredients should be entered into a pot with 12 rice-bowlsful of water, brought to the boil and simmered for 11.5 hours, until the volume of water has reduced down to 3 rice-bowlsful. The resulting decoction should be divided into 3 equal doses, one to be administered cold at 8 am before food, one at 3 pm, and the third in the evening before bed.

In serious cases, extra cinnamon bark should be ground to a fine powder and added to the already prepared decoction—the dose being 11.25 g/day. In patients where diminished platelet levels lead to excessive bruising, 11.25˜18.75 g of zeqi should be added to the prepared formula 2 decoction.

Formula 3: formula 3 comprises ginseng 11.25 g, fried baizhu 11.25 g, bai-fuling 11.25 g, gancao 11.25 g, danggui 11.25 g, chuanxiong 11.25 g, chi-shaoyao 11.25 g, gan-dihuang 11.25 g, bei-huangqi 37.5˜75 g, cinnamon bark 11.25˜18.75 g, dried ginger 11.25˜30 g, tianxiong 11.25˜30 g, mahuang 7.5˜15 g, dilong 11.25˜18.75 g, huangbo 11.25 g, lurong 11.25˜18.75 g and powdered chuanqi 11.25 g. This formula is suitable for use in the treatment of paralysis in patients where symptoms of poor appetite, hemiparalysis and impaired hematopoiesis form a pattern of Qi and Blood Deficiency with Yang Deficiency.

The method of preparation for formula 3 is as follows: the ginseng, cinnamon bark and chuanqi should first be machine-ground to a fine powder and put to one side, being added to the resultant decoction of the other ingredients. The baizhu, bai-fuling, gancao, chuanqiong, danggui, chi-shaoyao, gan-dihuang, huangqi, dried ginger and lurong should all be machine-sliced to a thickness of 0.15 cm, the mahuang and huangbo should be machine ground to grains 0.1 cm in diameter. The dilong should be cut into length of 1.5 cm. Finally the ingredients should be placed in a pot with 10 rice-bowlsfil of water, brought to the boil and simmered for 1˜1.5 hours, until the volume of water has reduced to 3 rice-bowlsful. Finally the aforementioned powdered ginseng, cinnamon bark and chuanqi should be mixed into the decoction, which should thereupon be divided into three equal portions. Each portion constitutes one dose, the first to be administered cold at 8 am before food, the second at 3 pm, and the third in the evening before bed.

In selection of the correct formula for the patient, formula 1 should be used in treatment of paralysis caused by viral infection, external injury, or abnormal cellular differentiation or of secondary induced paralysis in patients presenting with symptom patterns of Exterior Yang Deficiency with Cold Stagnation. Formula 2 should be used to treat paralysis in patients presenting with symptom patterns of Kidney Yang Deficiency with Cold Stagnation, and Formula 3 should be used for treatment of paralysis in patients presenting with symptom patterns of Qi and Blood Deficiency with Yang Deficiency.

Regardless of the formula used, if during the course of the therapy the symptom-pattern should change, then the formula should be changed accordingly. For example, if in the initial stage the diagnosis is trauma induced paralysis, then at that time, formula 1 should be administered; if the patients condition, during the course of the therapy should develop into a deficient kidney yang pattern with stagnant cold, then formula 2 should replace formula 1. Another patient may first present with a symptom-pattern of Qi and Blood Deficiency with Yang Deficiency, and accordingly be treated with formula 3, later if the symptom-pattern became one of viral infection, then the prescription should be changed to formula 1. Still further on in the course of treatment, if the symptom-pattern became one of Kidney Yang Deficiency with Cold Stagnation, then the formula prescription would now change to formula 2. In this way a full and unimpeded recovery may be achieved through application of this wide ranging Chinese herbal medicine composite system for use in treatment of paralysis.

Throughout the course of the above outlined special therapy using Chinese herbal medicine, the paralysed patient should expect to undergo various changes in sensory perception (particularly that of touch), as outlined below:

1. For a period the patient will undergo sensations of hourly alternating heat and cold. This symptom will subside automatically after about 2 weeks.

2. The symptom outlined above will recur at a rate somewhere in the region of once a fortnight to once every two months. After each of these periods, the patient will experience an improvement in tactile sensory awareness and motor control.

3. Uncontrollable spasms are an indication and result of maintained nerve signal transduction.

4. During the course of the return of tactile sensory awareness, the patient will undergo the following sequence of sensations: first from complete absence of tactile awareness, then to a feeling of great heaviness in the body and limbs as if one who wants to move but has no strength to do so, next will come a burning sensation in the parts of the body in contact with the bed. Next will come soreness, and tactile awareness of heavy pressure applied to the muscles affecting the deep nerves. Sensory awareness of lighter pressure applied to muscles affecting shallower nerves marks a further improvement in the patient's condition. Finally the patient will experience recovery of motor control.

5. The patient will experience a perceived band of tightness at the level of the spinal injury. For example with neck injury, the patient may complain of the sensation of having something stuck in the throat, unable to swallow it. Such sensations will migrate further down the body as the patient's condition improves. For example the patient there may next complain that the sensation of constriction has moved to the breast and corresponding spinal area. Again lower, the sensation will next manifest around the area of the diaphragm, then at the level of the umbilicus, followed by the groin, the buttocks—until a complete recovery is made.

6. From having absolutely no consciousness of urination, the condition will improve to a stage where the patient gains consciousness of the desire to urinate, further improving to a sensation akin to the a desire to ejaculate (NOTE: at this stage any male who has had a catheter inserted is prone to experience uncontrolled ejaculations). The next stage of improvement is marked by awareness of the urine as it passes through the urethra, then to the feeling of satisfaction experienced after urination, and finally to complete control over urination.

7. Consciousness of defecation will slowly improve from a state of absolute absence to a sensation of blockage, to burning sensations of the anus, next to awareness that stool has been passed, then to sensation of satisfaction on passing stool, and finally to complete control over defecation.

8. In cases of paralysis resulting from injury to the cells of the cervical spine, there will be impairment of the swallowing function. In the initial stages swallowing will be impossible, as if there were a cord pulled around the throat forbidding it. With improvement a small amount of the food to be swallowed would enter the stomach. At the next stage, swallowing would satisfactorily result in the food entering the stomach, but afterwards there would be an uncomfortable feeling in the throat. The next stage is marked by a feeling of something lodged in the throat—akin to the so-called “hysteria ball”. The final stage marks the complete return of the normal swallowing function with no discomfort.

9. Assorted sensations of being constricted with cord are likely to manifest around 34 weeks after commencing the treatment. Such sensations slowly diminish and finally disappear over a further period of 2˜3 weeks.

10. The male patient is likely to experience uncontrollable spontaneous erections, and may achieve erection as a result of stimulation, and hence may occasionally have intercourse. If the patient is fitted with a permanent catheter, he is prone to experience uncontrolled ejaculation, which if excessive could possibly lead to shock. In light of this danger it is advised that the permanent catheter be removed as early as possible. Even if the patient is able to achieve erection, he is likely to experience impaired sensation of the glans, possibly resulting in difficulty ejaculating.

11. The patient will experience uncontrollable spasm and contortion in the early stages. With improvement, such spasm will result only after touch stimulation, and with further improvement will diminish and disappear. The next stage is marked by improved control of joint movement, finally up to a state of completely natural movement.

12. Some joints may go through periods of hourly alternating hot and cold sensations, as well as those of distention and heaviness resembling symptoms of arthritis.

As a rule, during the course of treatment, a number of considerations should be noted, in order to ensure the patient's recovery progress without trouble or undue discomfort:

A—In the case of a patient who is conscious, but completely without feeling or control of the lower body, the greatest care should be taken when lifting the patient, in order to avoid adding to the injury, which might induce breathing difficulties leading to sudden death.

B—As far as possible the temptation to carry out spinal surgery such as spinal fixation, nerve transplant and spinal repair should be curbed. Only in situations of torn or severed spinal nerve should operating be considered.

C—The use of a bed fitted with automatic turning facility is advisable to limit the possibility of injury to the spinal column or nerves. Such injuries, resulting in breathing difficulties leading to sudden death, may occur as a result of the careworker's insufficient strength to turn the patient properly.

D—Spinal traction, manipulation, and stimulation, as well as spinal acupuncture and injection are all proscribed during the course of the treatment, as is massage of the joints, all of which could lead to breathing difficulties culminating in sudden death.

E—Upon injury, Chinese herbal medicine therapy should be commenced immediately.

As stated above, this innovation is a wide ranging and effective therapy for paralysis. In addition to its practical value and merits outlined above, this is an original and highly efficient natural method, as yet unprecedented by any similar product in its technical realm currently in the public domain. As such, the innovation fulfills the prerequisite criteria for the patent of an invention. And so, according to law it is beseeched that patent be granted for the above detailed innovation.

Appendix 1: Pinyin (romanization) and Latin terms for herbs

bai-fuling—sclerotium Poriae Cocos

baizhu—Rhizoma Atractylodis Macrocephalae

bei huangqi—Astragalus membraniceus (Fisch.) Bge

chi-shaoyao—Paeonia veitchii Lynch

chishao—Paeonia veitchii Lynch

chuanqi (powder)—Panax pseudoginseng (notoginseng) (pulverized root)

chuanxiong—Radix Ligustici Chuanxiong (Ligusticum wallichii Franch.)

cinnamon bark—Cortex cinnamomi Cassiae

danggui—Angelica sinesis (Oliv.) Diels (tails)

danggui tails—Angelica sinesis (Oliv.) Diels (tails)

dihuang—Radix Rehmanniae Glutinosae (raw)

dilong—Pheretima aspergillum (Perrier) or Allobophora calliginosa

dried ginger—Zingiber officinale Rose

duzhong—Eucommia ulmoides Oliv.

gancao—Glycyrhizae Uralensis, Radix

ginseng—Panax ginseng

gouqi-zi—Lycium Chinese Mull., fructus

honghua—Flos Carthami Tinctorri

huai-niuxi—Achyranthes bidentata B1.

huai-shanyao—Diorscorea opposite Thumb.

huangbo—Phollodendron Amurense Rupr.

huangqin—Scutellaria baicalensis Georgi.

lurong—Cervus Nippon Temminck or Cervus elaphus

mahuang—Ephedra sinica Stapf.

moyao—Commiphoramyrrha Engl., (Myrrha)

ruxiang—Bosweellia carterii Birdw, (Gummi Olibanum)

shan-zhuyu—Cornus officinalis Sied. Et. Zucc.

taoren—Prunus persica (L.) (semen)

tianxiong—Aconitum carmichaeli Debx. (prepaired)

tusi-zi—Cuscutae Chinesis (semen)

Appendix 2: measurements

1 rice-bowl 250 cc.

I claim:

1. A Chinese herbal medicine composite system for use in treatment of paralysis, comprising herbal formulas formula 1, formula 2 and formula 3, formula 1 being suitable for treatment of paralysis caused by viral infection, external injury, or abnormal cellular differentiation or secondary induced paralysis in patients presenting with symptom patterns of Exterior Yang Deficiency with Cold Stagnation, formula 2 being suitable for treatment of paralysis in patients presenting with symptom patterns of Kidney Yang Deficiency with Cold Stagnation, and Formula 3 being suitable for treatment of paralysis in patients presenting with symptom patterns of Qi and Blood Deficiency with Yang Deficiency;
Formula 1: formula 1 comprises: mahuang 7.5˜15 g, cinnamon bark 11.25˜18.75 g, huangqin 11.25 g, honghua 11.25 g, dilong 11.25˜18.75 g, danggui tails 11.25 g, taoren paste 11.25 g, gancao 11.25 g, chuanxiong 11.25 g, chishao 11.25 g, bei huangqi 37.5 g, ruxiang 11.25 g, moyao 11.25 g, dried ginger 11.25˜18.75 g, tianxiong 11.25˜18.75 g, and chuanqi powder 11.25 g, this formula being for use in treatment of paralysis caused by viral infection, external injury, or abnormal cellular differentiation or of secondary induced paralysis in patients presenting with symptom patterns of Exterior Yang Deficiency with Cold Stagnation.
Formula 2: formula 2 comprises dry dihuang 15 g, shan-zhuyu, 15 g, huai-shanyao 15 g, bai-fuling 15 g, tusi-zi 15 g, huai-niuxi 15 g, danggui 15 g, lurong 15 g, gouji-zi 11.25 g, fried duzhong 11.25 g, mahuang 11.25 g, huangbo 11.25 g, dried ginger 18.75 g˜26.25 g, tianxiong 18.75˜26.25 g, and cinnamon bark 18.25 g, this formula being suitable for use in the treatment of paralysis where symptoms of low blood pressure, osteoporosis, atrophy of the liver, atrophy of the kidney, coldness in the limbs, low body temperature, and or constipation form a pattern of Kidney Yang Deficiency with Cold Stagnation.
Formula 3: formula 3 comprises ginseng 11.25 g, fried baizhu 11.25 g, bai-fuling 11.25 g, gancao 11.25 g, danggui 11.25 g, chuanxiong 11.25 g, chi-shaoyao 11.25 g, gan-dihuang 11.25 g, bei-huangqi 37.5˜75 g, cinnamon bark 11.25˜18.75 g, dried ginger 11.25˜30 g, tianxiong 11.25˜30 g, mahuang 7.5˜15 g, dilong 11.25˜18.75 g, huangbo 11.25 g, lurong 11.25˜18.75 g and powdered chuanqi 11.25 g, this formula being suitable for use in the treatment of paralysis in patients where symptoms of poor appetite, hemiparalysis and impaired hematopoiesis form a pattern of Qi and Blood Deficiency with Yang Deficiency.
whereby taken as a whole, the above comprises a Chinese herbal medicine composite system suitable for wide ranging use in treatment of paralysis.

2. The Chinese herbal medicine composite system for use in treatment of paralysis as claimed in clam 1, wherein the method of preparation of the therein mentioned formula 1 is as follows:
the mahuang, cinnamon bark, huangqin, danggui tails, chishao and tianxiong should all be cut into small pieces or grains 0.1˜0.3 cm in diameter using a suitable herb cutting machine, the dilong should be cut into lengths of 1.5 cm, the huangqi and dried ginger should be sliced into pieces 0.15 thick, the chuanqi should be machine-ground to a fine powder, the gancao should be sliced into pieces 0.15 in thickness. The ruxiang and moyao should be crystal-cut into 1 cm³ cubes, the above ingredients, excluding the powdered chuanqi, along with an amount of water equivalent to 8 rice-bowlsfil, should be entered into a pot and brought to the boil, and subsequently simmered for 1˜1.5 hours, and when the amount of water has reduced to the equivalent of 3 bowlsful, the powdered chuanqi should be added.

3. The Chinese herbal medicine composite system for use in treatment of paralysis, wherein to the therein mentioned formula 1, ginseng may be added at a dosage of 11.25 g daily. Ideally the ginseng should be masticated slowly before swallowing.

4. The Chinese herbal medicine composite system for use in treatment of paralysis as claimed in claim 1, wherein ginseng and chuanqi may be ground to a fine powder and mixed into the prepared decoction. Cinnamon bark may also be ground to a fine powder and added to the prepared decoction, and need not be cooked.

5. The Chinese herbal medicine composite system for use in treatment of paralysis as claimed in claim 1, wherein the method of preparation of the therein mentioned formula 2 is as follows:
the dihuang, huai-shanyao, bai-fuling, danggui, crude lurong and dried ginger should all be machine-cut to a thickness of 0.15 cm, the huai-niuxi should be cut into lengths of 1 cm, the duzhong should first be fried lightly; frying only until it starts to give off a pungent aroma and white smoke, the duzhong should then be immersed in salt water, and left until the water has soaked right into the material, thereupon being retrieved from the water and fried on a low heat until dry to the touch, and consequently the duzhong should be placed in an oven at 50° C., until completely dry, the danggui should be sprayed or dipped in rice wine, which should be allowed to soak right through the tissue of the material, the danggui should then be sliced into strips which are then to be placed in a low temperature oven (40° C.), until the moisture has evaporated, a pestle and mortar should be used to smash the outer skin of the gouji-zi, the mahuang, tianxiong, cinnamon bark and huangbo should all be machine-ground into grains of 0.1˜0.15 cm in diameter, the tusi-zi should be stir-fried until the outer casing bursts open, the shan-zhuyu should first be washed and dried, whereupon the flowers, seeds and sterns should be removed, and finally all the ingredients should be entered into a pot with 12 rice-bowlsful of water, brought to the boil and simmered for 1˜1.5 hours, until the volume of water has reduced down to 3 rice-bowlsful.

6. The Chinese herbal medicine composite system for use in treatment of paralysis as claimed in claim 1, wherein to the therein mentioned formula 2, cinnamon bark may be added after being ground to a fine powder and at a dosage of 11.25 g daily. In such case, the ground cinnamon bark should be added only after decoction of the aforementioned formula.

7. The Chinese herbal medicine composite system for use in treatment of paralysis as claimed in claim 1, wherein to the therein mentioned formula 2 may be added zexi at a dosage of 11.25˜18.75 g daily, in such cases as the paralysed patient experiences excessive subcutaneous bruising as a result of diminished platelet levels.

8. The Chinese herbal medicine composite system for use in treatment of paralysis as claimed in claim 1, wherein the method of preparation of the therein mentioned formula 3 is as follows:
the ginseng, cinnamon bark and chuanqi should first be machine-ground to a fine powder and put to one side, being added to the resultant decoction of the other ingredients, the baizhu, bai-fuling, gancao, chuanqiong, danggui, chi-shaoyao, gan-dihuang, huangqi, dried ginger and lurong should all be machine-sliced to a thickness of 0.15 cm, the mahuang and huangbo should be machine ground to grains 0.1 cm in diameter, the dilong should be cut into length of 1.5 cm, and the ingredients should be placed in a pot with 10 rice-bowlsful of water, brought to the boil and simmered for 1˜1.5 hours, until the volume of water has reduced to 3 bowlsful, and finally the aforementioned powdered ginseng, cinnamon bark and chuanqi should be mixed into the decoction.

9. The Chinese herbal medicine composite system for use in treatment of paralysis as claimed in claim 1, wherein the important ingredients are: mahuang 7.5˜15 g, cinnamon bark 11.25˜18.75 g, huangqin 11.25 g, honghua 11.25 g, dilong 11.25˜18.75 g, danggui tails 11.25 g, taoren paste 11.25 g, gancao 11.25 g, chuanxiong 11.25 g, chishao 11.25 g, bei huangqi 37.5 g, ruxiang 11.25 g, moyao 11.25 g, dried ginger 11.25˜18.75 g, tianxiong 11.25˜18.75 g, and chuanqi powder 11.25 g, and this formula is for use in treatment of paralysis caused by viral infection, external injury, or abnormal cellular differentiation or of secondary induced paralysis in patients presenting with symptom patterns of Exterior Yang Deficiency with Cold Stagnation.

10. The Chinese herbal medicine composite system for use in treatment of paralysis as claimed in claim 1, wherein preparation of the materials therein mentioned are as follows: the mahuang, cinnamon bark, huangqin, danggui tails, chishao and tianxiong should all be cut into small pieces or grains 0.1˜0.3 cm in diameter using a suitable herb cutting machine, the dilong should be cut into lengths of 1.5 cm, the huangqi and dried ginger should be sliced into pieces 0.15 thick, the chuanqi should be machine-ground to a fine powder, the gancao should be sliced into pieces 0.15 in thickness, the ruxiang and moyao should be crystal-cut into 1 cm³ cubes, the above ingredients, excluding the powdered chuanqi, along with an amount of water equivalent to 8 rice-bowlsful, should be entered into a pot and brought to the boil, and subsequently simmered for 1˜1.5 hours, and when the amount of water has reduced to the equivalent of 3 bowlsful, the powdered chuanqi should be added.

11. The Chinese herbal medicine composite system for use in treatment of paralysis as claimed in claim 1, wherein to the therein mentioned ingredients may be added ginseng at a dosage of 11.25 g daily, best administered by masticating slowly before swallowing.

12. The Chinese herbal medicine composite system for use in treatment of paralysis as claimed in claim 1, wherein to the therein mentioned ginseng may be added powdered chuanqi, which after having been ground together to a fine powder, should be mixed into the already prepared decoction. In addition, cinnamon bark may also be ground to a fine powder and added to the already ground decoction, not requiring to be cooked.

13. The Chinese herbal medicine composite system for use in treatment of paralysis as claimed in claim 12, wherein the important ingredients are: dihuang 15 g, shan-zhuyu, 15 g, huai-shanyao 15 g, bai-fuling 15 g, tusi-zi 15 g, huai-niuxi 15 g, danggui 15 g, lurong 15 g, gouji-zi 11.25 g, fried duzhong 11.25 g, mahuang 11.25 g, huangbo 11.25 g, dried ginger 18.75 g˜26.25 g, tianxiong 18.75˜26.25 g, and cinnamon bark 18.25 g. This formula is suitable for use in the treatment of paralysis where symptoms of low blood pressure, osteoporosis, atrophy of the liver, atrophy of the kidney, coldness in the limbs, low body temperature, and or constipation form a pattern of Kidney Yang Deficiency with Cold Stagnation.

14. The Chinese herbal medicine composite system for use in treatment of paralysis as claimed in claim 13, wherein preparation of the materials therein mentioned are as follows: the dihuang, huai-shanyao, bai-fuling, danggui, crude lurong and dried ginger should all be machine-cut to a thickness of 0.15 cm, the huai-niuxi should be cut into lengths of 1 cm, the duzhong should first be fried lightly; frying only until it starts to give off a pungent aroma and white smoke, the duzhong should then be immersed in salt water, and left until the water has soaked right into the material, thereupon being retrieved from the water and fried on a low heat until dry to the touch, consequently the duzhong should be placed in an oven at 50° C., until completely dry, the danggui should be sprayed or dipped in rice wine, which should be allowed to soak right through the tissue of the material, the danggui should then be sliced into strips which are then to be placed in a low temperature oven (40° C.), until the moisture has evaporated, a pestle and mortar should be used to smash the outer skin of the gouji-zi, the mahuang, tianxiong, cinnamon bark and huangbo should all be machine-ground into grains of 0.1˜0.15 cm in diameter, the tusi-zi should be stir-fried until the outer casing bursts open, the shan-zhuyu should first be washed and dried, whereupon the flowers, seeds and stems should be removed, and finally all the ingredients should be entered into a pot with 12 bowlsful of water, brought to the boil and simmered for 1˜1.5 hours, until the volume of water has reduced down to 3 bowlsful.

15. The Chinese herbal medicine composite system for use in treatment of paralysis as claimed in claim 13, wherein to the materials therein mentioned, after their preparation and decoction, may be added cinnamon bark, such as has been ground to a fine powder, it not requiring to be cooked.

16. The Chinese herbal medicine composite system for use in treatment of paralysis as claimed in claim 13, wherein to the materials therein mentioned, may be added zeqi at a dosage of 11.25˜18.75 g daily, in such case as the paralysed patient suffers excessive subcutaneous bruising as a result of diminished platelet levels.

17. The Chinese herbal medicine composite system for use in treatment of paralysis as claimed in claim 13, wherein the important ingredients of the Chinese herbal medicine composite system for use in treatment of paralysis are: ginseng 11.25 g, fried baizhu 11.25 g, bai-fuling 11.25 g, gancao 11.25 g, danggui 11.25 g, chuanxiong 11.25 g, chi-shaoyao 11.25 g, gan-dihuang 11.25 g, bei-huangqi 37.5˜75 g, cinnamon bark 11.25˜18.75 g, dried ginger 11.25˜30 g, tianxiong 11.25˜30 g, mahuang 7.5˜15 g, dilong 11.25˜18.75 g, huangbo 11.25 g, lurong 11.25˜18.75 g and powdered chuanqi 11.25 g, this formula is suitable for use in the treatment of paralysis in patients where symptoms of poor appetite, hemiparalysis and impaired hematopoiesis form a pattern of Qi and Blood Deficiency with Yang Deficiency.

18. The Chinese herbal medicine composite system for use in treatment of paralysis as claimed in claim 17, wherein preparation of the materials therein mentioned are as follows: the ginseng, cinnamon bark and chuanqi should first be machine-ground to a fine powder and put to one side, being added to the resultant decoction of the other ingredients. The baizhu, bai-fuling, gancao, chuanqiong, danggui, chi-shaoyao, gan-dihuang, huangqi, dried ginger and lurong should all be machine-sliced to a thickness of 0.15 cm, the mahuang and huangbo should be machine ground to grains 0.1 cm in diameter, the dilong should be cut into length of 1.5 cm, finally the ingredients should be placed in a pot with 10 rice-bowlsful of water, brought to the boil and simmered for 1˜1.5 hours, until the volume of water has reduced to 3 rice-bowlsful, and finally the aforementioned powdered ginseng, cinnamon bark and chuanqi should be mixed into the decoction.
